(19) United States
(12) Patent Application Publication
(10) Pub. No.: US 2008/0242869 A1
(43) Pub. Date: Oct. 2, 2008

(54) Tri(Cyclo) Substituted Amide Compounds

(76) Inventor: Matthew Fyfe, Oxford (GB)

Correspondence Address: OSI PHARMACEUTICALS, INC., 41 PINELAWN ROAD, MELVILLE, NY, 11747, US

(21) Appl. No.: 11/578,752

(22) PCT Filed: Apr. 19, 2005
(86) PCT No.: PCT/GB2005/050053

§ 371 Date: Nov. 5, 2007

Related U.S. Application Data
(60) Provisional application No. 60/601,077, filed on Aug. 12, 2004.
(60) Provisional application No. 60/564,171, filed on Apr. 21, 2004.

(51) Int. Cl. C07D 277/40

(52) U.S. Cl. 548/204

(57) ABSTRACT

Compounds of Formula (I) or pharmaceutically acceptable salts thereof, are useful in the prophylactic and therapeutic treatment of hyperglycemia and diabetes.

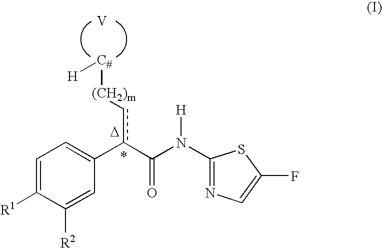

### BACKGROUND OF THE INVENTION

The present invention is directed to tri(cyclo) substituted amide compounds. In particular, the present invention is directed to amide compounds substituted i) at the carbonyl carbon with an ethyl/ethenyl attached to a phenyl ring and a carbocyclic ring, and ii) at the amino with a fluoro substituted thiazole ring, which are modulators of glucokinase and are useful in the prophylactic or therapeutic treatment of hyperglycemia and diabetes, particularly type II diabetes.

Glucokinase (“GK”) is believed to be important in the body's regulation of its plasma glucose level. GK, found principally in the liver and pancreas, is one of four hexokinases that catalyze the initial metabolism of glucose. The GK pathway is saturated at higher glucose levels than the other hexokinase pathways (See R. L. Printz et al., Annu. Rev. Nutr., 13:463-496 (1993)). GK is critical to maintaining the glucose balance in mammals. Animals that do not express GK die soon after birth with diabetes, while animals that overexpress GK have improved glucose tolerance. Activation of GK can lead to hyperinsulinemic hypoglycemia. (See, for example, H. B. T. Christesen et al., Diabetes, 51:1240-1246 (2002)). Additionally, type II maturity-onset diabetes of the young is caused by the loss of function mutations in the GK gene, suggesting that GK operates as a glucose sensor in humans (Y. Liang et al., Biochem. J. 309:167-173 (1995)). Thus, compounds that activate GK increase the sensitivity of the GK sensory system and would be useful in the treatment of hyperglycemia—particularly the hyperglycemia associated with type II diabetes. It is therefore desirable to provide novel compounds that activate GK to treat diabetes.

International Patent Publication No. WO2001/044216 and U.S. Pat. No. 6,353,111 describe (E)-2,3-disubstituted-N-heteroarylacrylamides as GK activators. International Patent Publication No. WO2002/014312 and U.S. Pat. Nos. 6,369,232, 6,388,088 and 6,441,180 describe tetrazolylphenylacetamide GK activators. International Patent Publication No. WO2000/058293, European Patent Application No. EP 1169312 and U.S. Pat. No. 6,320,050 describe arylcycloalkylpropionamide GK activators. International Patent Publication No. WO2002/008209 and U.S. Pat. No. 6,486,184 describe alpha-acyl and alpha-heteroatom-substituted benzene acetamide GK activators as anti-diabetic agents. International Patent Publication No. WO2001/083478 describes hydantoin-containing GK activators. International Patent Publication No. WO2001/083465 and U.S. Pat. No. 6,388,071 describe alkynylphenyl heteroaromatic GK activators. International Patent Publication No. WO2001/085707 and U.S. Pat. No. 6,489,485 describe para-amine substituted phenylamide GK activators. International Patent Publication No. WO2002/046173 and U.S. Pat. Nos. 6,433,188, 6,441,184 and 6,448,399 describe fused heteroaromatic GK activators. International Patent Publication No. WO2002/048106 and U.S. Pat. No. 6,482,951 describe isoindolin-1-one GK activators. International Patent Publication No. WO2001/085706 describes substituted phenylacetamide GK activators for treating type II diabetes. U.S. Pat. No. 6,384,220 describes para-aryl or heteroaryl substituted phenyl GK activators. French Patent No. 2,834,295 describes methods for the purification and crystal structure of human GK. International Patent Publication No. WO2003/095438 describes N-heteroaryl phenylacetamides and related compounds as GK activators for the treatment of type II diabetes. U.S. Pat. No. 6,610,846 describes the preparation of cycloalkylheteroaryl propionamides as GK activators. International Patent Publication No. WO2003/000262 describes vinyl phenyl GK activators. International Patent Publication No. WO2003/000267 describes aminonicotinate derivatives as GK modulators. International Patent Publication No. WO2003/015774 describes compounds as GK modulators. International Patent Publication No. WO2003/047626 describes the use of a GK activator in combination with a glucagon antagonist for treating type II diabetes. International Patent Publication No. WO2003/055482 describes amide derivatives as GK activators. International Patent Publication No. WO2003/080585 describes aminobenzamide derivatives with GK activity for the treatment of diabetes and obesity. International Patent Publication No. WO2003/097824 describes human liver GK crystals and their used for structure-based drug design. International Patent Publication No. WO2004/002481 discloses arylcarbonyl derivatives as GK activators. International Patent Publication Nos. WO2004/072031 and WO2004/072066 (published after the priority date of the present application) discloses various tri(cyclo) substituted amide compounds which are modulators of glucokinase.

### SUMMARY OF THE INVENTION

Compounds represented by Formula (I):

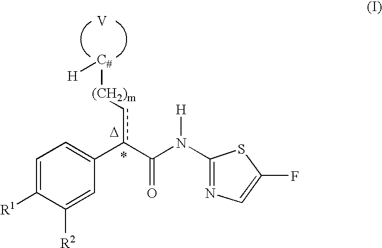

or pharmaceutically acceptable salts thereof, are useful in the prophylactic or therapeutic treatment of hyperglycemia and diabetes, particularly type II diabetes.

### DETAILED DESCRIPTION OF THE INVENTION

The present invention is directed to a compound of Formula (I):

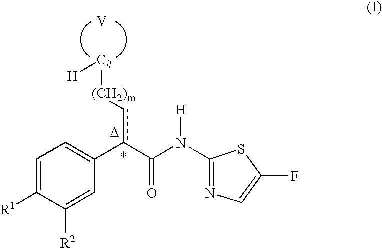

or a pharmaceutically acceptable salt thereof, wherein:

V is (CH₂)ₖ where one CH₂ group may optionally be replaced by CH(OH), C═O, C═NOH, C═NOCH₃, CHX, CXX¹, CH(OCH₃), CH(OCOCH₃), CH(C₁₋₄alkyl), or C(OH)(C₁₋₄alkyl);

X and X¹ are independently selected from fluoro and chloro;

R¹ and R² are independently selected from hydrogen, halogen, hydroxy, amino, cyano, nitro, SR³, SOR³, SO₂R³, SO₂NR⁴R⁵, NHSO₂R³, or a C₁₋₄alkyl, C₂₋₄alkenyl, C₂₋₄alkynyl, C₁₋₄alkoxy, or heteroaryl group, wherein any group is optionally substituted with 1 to 5 substituents independently selected from halogen, cyano, nitro, hydroxy, C₁₋₂alkoxy, —N(C₀₋₂alkyl)(C₀₋₂alkyl), C₁₋₂alkyl, CFₙH₃₋ₙ, aryl, heteroaryl, —CON(C₀₋₂alkyl)(C₀₋₂alkyl), SCH₃, SOCH₃, SO₂CH₃, and —SO₂N(C₀₋₂alkyl)(C₀₋₂alkyl);

R³ is a C₁₋₄alkyl group, C₃₋₇cycloalkyl group, aryl group, heteroaryl group, or 4- to 7-membered heterocyclic group, wherein any group is optionally substituted with 1 to 5 substituents independently selected from halogen, cyano, nitro, hydroxy, C₁₋₂alkoxy, —N(C₀₋₂alkyl)(C₀₋₂alkyl), C₁₋₂alkyl, C₃₋₇cycloalkyl, 4- to 7-membered heterocyclic ring, CFₙH₃₋ₙ, aryl, heteroaryl, COC₁₋₂alkyl, —CON(C₀₋₂alkyl)(C₀₋₂alkyl), SOCH₃, SO₂CH₃, and —SO₂N(C₀₋₂alkyl)(C₀₋₂alkyl);

R⁴ and R⁵ are independently hydrogen, or a C₁₋₄alkyl group, C₃₋₇cycloalkyl group, aryl group, heteroaryl group, or 4- to 7-membered heterocyclic group, wherein any group is optionally substituted with 1 to 5 substituents independently selected from halogen, cyano, nitro, hydroxy, C₁₋₂alkoxy, —N(C₀₋₂alkyl)(C₀₋₂alkyl), C₁₋₂alkyl, C₃₋₇cycloalkyl, 4- to 7-membered heterocyclic ring, CFₙH₃₋ₙaryl, heteroaryl, —CON(C₀₋₂alkyl)(C₀₋₂alkyl), SOCH₃, SO₂CH₃, and —SO₂N(C₀₋₂alkyl)(C₀₋₂alkyl);

or R⁴ and R⁵ together form a 4- to 8-membered heterocyclic ring which is optionally substituted with 1 or 2 substituents independently selected from C₁₋₂alkyl and hydroxy;

k is an integer from 2 to 7;

m is 0 or 1;

n is 1, 2 or 3; and

the dotted line together with the solid line forms an optional double bond, and Δ indicates that the double bond has the (E)-configuration.

If the dotted line together with the solid line forms a single bond, the carbon atom linking the aryl ring and —HC<>V-containing sidechain to the amide carbonyl carbon, i.e. the carbon atom labelled with “*”, is a chiral centre. Accordingly, at this centre, the compound may be present either as a racemate or as a single enantiomer in the (R)— or (S)-configuration. The (R)-enantiomers are preferred. The carbon atom labelled with “#” may also be chiral. Accordingly, at this centre, the compound may be present either as a racemate or as a single enantiomer in the (R)— or (S)-configuration. The (R)-enantiomers are preferred when the dotted line together with the solid line represents a single bond. When the dotted line together with the solid line forms a double bond, the (S)-enantiomers are preferred.

In a further aspect, the present invention is directed to a compound represented by Formula (Ia):

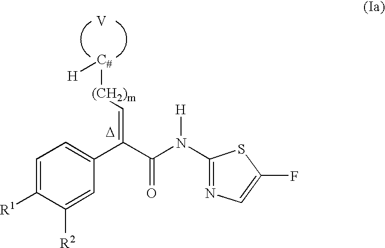

or a pharmaceutically acceptable salt thereof, wherein V, R¹, R², m and Δ are as defined above in Formula (I).

In another embodiment, the present invention is directed to a compound represented by Formula (Ia), or a pharmaceutically acceptable salt thereof, wherein the group formed by —HC< and >V represents oxocycloalkyl or hydroxycycloalkyl, e.g. 3-oxocyclopentyl particularly (R)-3-oxocyclopentyl, 4-oxocyclohexyl or 3-hydroxycyclopentyl, especially (R)-3-oxocyclopentyl.

In a further and preferred aspect, the present invention is directed to a compound represented by Formula (Ib):

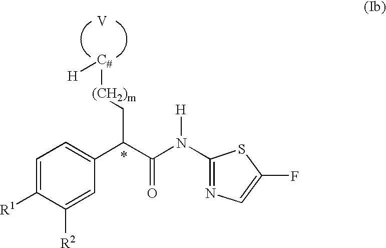

or a pharmaceutically acceptable salt thereof, wherein V, R¹, R² and m are as defined above in Formula (I).

In an embodiment of this preferred aspect, the present invention is directed to a compound represented by Formula (Ib), or a pharmaceutically acceptable salt thereof, wherein the group formed by —HC< and >V represents oxocycloalkyl or hydroxycycloalkyl, e.g. 3-oxocyclopentyl particularly (R)-3-oxocyclopentyl, 4-oxocyclohexyl or 3-hydroxycyclopentyl, especially (R)-3-oxocyclopentyl.

The molecular weight of the compounds of Formula (I) is preferably less than 800, more preferably less than 600, most preferably less than 500.

In the present invention, R¹ and R² are preferably not both hydrogen.

In the present invention, R¹ is preferably CF₃, SOR³, SO₂R³, SO₂NR⁴R⁵, NHSO₂R³, and triazolyl; more preferably SOR³, SO₂R³, or SO₂NR⁴R⁵; most preferably SO₂R³ or SO₂NR⁴R⁵, especially SO₂R³.

In particular R¹ is SO₂C₃₋₄cycloalkyl, especially SO₂cyclopropyl.

In the present invention, R² is preferably hydrogen, chloro, fluoro, or trifluoromethyl; more preferably hydrogen or chloro.

In the present invention, R³ is preferably C₁₋₃alkyl or C₃₋₄cycloalkyl, more preferably C₃₋₄cycloalkyl, especially cyclopropyl.

In the present invention, R⁴ and R⁵ are preferably independently hydrogen or C₁₋₄alkyl, e.g. one of R⁴ and R⁵ is hydrogen and the other is ethyl, or combine to form a 4- to 8-membered heterocyclic ring. R⁴ and R⁵ are preferably not both hydrogen.

In the present invention, m is preferably 0.

In the present invention V is preferably (CH₂)ₖ where one CH₂ group is replaced by CH(OH) or C═O.

In the present invention, k is preferably 4 or 5.

Specific compounds of the invention which may be mentioned are:

2(R)-2-(3-Chloro-4-methanesulfonylphenyl)-N-(5-fluorothiazol-2-yl)-3-((R)-3-oxocyclopentyl)propionamide;

2(R)-2-(3-Chloro-4-methanesulfonylphenyl)-N-(5-fluorothiazol-2-yl)-3-(4-oxocyclohexyl)propionamide;

2(R)-2-(3-Chloro-4-methanesulfonylphenyl)-N-(5-fluorothiazol-2-yl)-3-(3-hydroxycyclopentyl)propionamide;

(E)-N-(5-Fluorothiazol-2-yl)-2-(4-methanesulfonylphenyl)-3-((S)-3-oxocyclopentyl)acrylamide;

(E)-N-(5-Fluorothiazol-2-yl)-2-(4-methanesulfonylphenyl)-3-(4-oxocyclohexyl)acrylamide;

(E)-N-(5-Fluorothiazol-2-yl)-3-(3-hydroxycyclopentyl)-2-(4-methanesulfonylphenyl)acrylamide;

2(R)-2-(4-Cyclopropanesulfonylphenyl)-N-(5-fluorothiazol-2-yl)-3-((R)-3-oxocyclopentyl)propionamide;

2(R)-2-(4-Cyclopropanesulfonylphenyl)-N-(5-fluorothiazol-2-yl)-3-(4-oxocyclohexyl)propionamide;

2(R)-2-(4-Cyclopropanesulfonylphenyl)-N-(5-fluorothiazol-2-yl)-3-(3-hydroxycyclopentyl)propionamide;

2(R)-2-(4-Cyclobutanesulfonylphenyl)-N-(5-fluorothiazol-2-yl)-3-(3-oxocyclopentyl)propionamide;

2(R)-2-(4-Cyclobutanesulfonylphenyl)-N-(5-fluorothiazol-2-yl)-3-(4-oxocyclohexyl)propionamide;

2(R)-2-(4-Cyclobutanesulfonylphenyl)-N-(5-fluorothiazol-2-yl)-3-(3-hydroxycyclopentyl)propionamide;

2(R)-2-(3-Fluoro-4-methanesulfonylphenyl)-N-(5-fluorothiazol-2-yl)-3-((R)-3-oxocyclopentyl)propionamide;

2(R)-2-(3-Fluoro-4-methanesulfonylphenyl)-N-(5-fluorothiazol-2-yl)-3-(4-oxocyclohexyl)propionamide; and

2(R)-2-(3-Fluoro-4-methanesulfonylphenyl)-N-(5-fluorothiazol-2-yl)-3-(3-hydroxycyclopentyl)propionamide;

or a pharmaceutically acceptable salt of any one thereof.

While the preferred groups for each variable have generally been listed above separately for each variable, preferred compounds of this invention include those in which several or each variable in Formula (I) is selected from the preferred, more preferred, most preferred, especially or particularly listed groups for each variable. Therefore, this invention is intended to include all combinations of preferred, more preferred, most preferred, especially and particularly listed groups.

As used herein, unless stated otherwise, “alkyl” as well as other groups having the prefix “alk” such as, for example, alkoxy, alkenyl, alkynyl, and the like, means carbon chains which may be linear or branched or combinations thereof. Examples of alkyl groups include methyl, ethyl, propyl, isopropyl, butyl, sec- and tert-butyl, pentyl, hexyl, heptyl and the like. “Alkenyl”, “alkynyl” and other like terms include carbon chains having at least one unsaturated carbon-carbon bond.

As used herein, for example, “C₀₋₄alkyl” is used to mean an alkyl having 0-4 carbons—that is, 0, 1, 2, 3, or 4 carbons in a straight or branched configuration. An alkyl having no carbon is hydrogen when the alkyl is a terminal group. An alkyl having no carbon is a direct bond when the alkyl is a bridging (connecting) group.

The terms “cycloalkyl” and “carbocyclic ring” mean carbocycles containing no heteroatoms, and includes monocyclic saturated C₃₋₇carbocycles. Examples of cycloalkyl and carbocyclic rings include cyclopropyl, cyclobutyl, cyclopentyl and cyclohexyl and the like.

The term “halogen” includes fluorine, chlorine, bromine, and iodine atoms.

The term “aryl” includes, for example, phenyl and naphthyl, preferably phenyl.

Unless otherwise stated, the term “heterocyclic ring” includes 4- to 8-membered saturated rings containing one or two heteroatoms selected from oxygen, sulfur and nitrogen. The heteroatoms are not directly attached to one another. Examples of heterocyclic rings include oxetane, tetrahydrofuran, tetrahydropyran, oxepane, oxocane, thietane, tetrahydrothiophene, tetrahydrothiopyran, thiepane, thiocane, azetidine, pyrrolidine, piperidine, azepane, azocane, [1,3]dioxane, oxazolidine, piperazine, and the like. Other examples of heterocyclic rings include the oxidised forms of the sulfur-containing rings. Thus, tetrahydrothiophene 1-oxide, tetrahydrothiophene 1,1-dioxide, tetrahydrothiopyran 1-oxide, and tetrahydrothiopyran 1,1-dioxide are also considered to be heterocyclic rings.

Unless otherwise stated, the term “heteroaryl” includes 5- or 6-membered heteroaryl rings containing 1-4 heteroatoms selected from oxygen, sulfur and nitrogen. Examples of such heteroaryl rings are furyl, thienyl, pyrrolyl, pyrazolyl, imidazolyl, oxazolyl, isoxazolyl, thiazolyl, isothiazolyl, triazolyl, oxadiazolyl, thiadiazolyl, tetrazolyl, pyridinyl, pyridazinyl, pyrimidinyl, pyrazinyl and triazinyl.

The above formulae are shown without a definitive stereochemistry at certain positions. The present invention includes all stereoisomers (e.g. geometric isomers, optical isomers, diastereoisomers, etc.) and pharmaceutically acceptable salts thereof, except where specifically drawn or stated otherwise. Further, mixtures of stereoisomers as well as isolated specific stereoisomers are also included, except where specifically drawn or stated otherwise. During the course of the synthetic procedures used to prepare such compounds, or in using racemization or epimerization procedures known to those skilled in the art, the products of such procedures can be a mixture of stereoisomers. When a tautomer of the compound of the above formulae exists, the present invention includes any possible tautomers and pharmaceutically acceptable salts thereof, and mixtures thereof, except where specifically drawn or stated otherwise. When the compound of the above formulae and pharmaceutically acceptable salts thereof exist in the form of solvates or polymorphic forms, the present invention includes any possible solvates and polymorphic forms. The type of a solvent that forms the solvate is not particularly limited so long as the solvent is pharmacologically acceptable. For example, water, ethanol, propanol, acetone or the like can be used.

Since the compounds of Formula (I) are intended for pharmaceutical use they are preferably provided in substantially pure form, for example at least 60% pure, more suitably at least 75% pure, at least 95% pure and especially at least 98% pure (% are on a weight for weight basis).

The invention also encompasses a pharmaceutical composition that is comprised of a compound of Formula (I), or a pharmaceutically acceptable salt thereof, in combination with a pharmaceutically acceptable carrier.

Preferably the composition is comprised of a pharmaceutically acceptable carrier and a non-toxic therapeutically effective amount of a compound of Formula (I), or a pharmaceutically acceptable salt thereof.

Moreover, within this embodiment, the invention encompasses a pharmaceutical composition for the prophylaxis or treatment of hyperglycemia and diabetes, particularly type II diabetes, by the activation of GK, comprising a pharmaceutically acceptable carrier and a non-toxic therapeutically effective amount of compound of Formula (I), or a pharmaceutically acceptable salt thereof.

The invention also provides the use of a compound of Formula (I), or a pharmaceutically acceptable salt thereof as a pharmaceutical.

The compounds and compositions of the present invention are effective for treating hyperglycemia and diabetes, particularly type II diabetes, in mammals such as, for example, humans.

The invention also provides a method of prophylactic or therapeutic treatment of a condition where activation of GK is desirable comprising a step of administering an effective amount of a compound of Formula (I), or a pharmaceutically acceptable salt thereof.

The invention also provides a method of prophylactic or therapeutic treatment of hyperglycemia or diabetes, particularly type II diabetes, comprising a step of administering an effective amount of a compound of Formula (I), or a pharmaceutically acceptable salt thereof.

The invention also provides a method of prevention of diabetes, particularly type II diabetes, in a human demonstrating pre-diabetic hyperglycemia or impaired glucose tolerance comprising a step of administering an effective prophylactic amount of a compound of Formula (I), or a pharmaceutically acceptable salt thereof.

The invention also provides the use of a compound of Formula (I), or a pharmaceutically acceptable salt thereof, as a GK activator.

The invention also provides the use of a compound of Formula (I), or a pharmaceutically acceptable salt thereof, for the prophylactic or therapeutic treatment of hyperglycemia or diabetes, particularly type II diabetes.

The invention also provides the use of a compound of Formula (I), or a pharmaceutically acceptable salt thereof, for the prevention of diabetes, particularly type II diabetes, in a human demonstrating pre-diabetic hyperglycemia or impaired glucose tolerance.

The invention also provides the use of a compound of Formula (I), or a pharmaceutically acceptable salt thereof, in the manufacture of a medicament for the activation of GK.

The invention also provides the use of a compound of Formula (I), or a pharmaceutically acceptable salt thereof, in the manufacture of a medicament for the prophylactic or therapeutic treatment of hyperglycemia or diabetes, particularly type II diabetes.

The invention also provides the use of a compound of Formula (I), or a pharmaceutically acceptable salt thereof, in the manufacture of a medicament for the prevention of diabetes, particularly type II diabetes, in a human demonstrating pre-diabetic hyperglycemia or impaired glucose tolerance.

The compounds and compositions of the present invention may be optionally employed in combination with one or more other anti-diabetic agents or anti-hyperglycemic agents, which include, for example, sulfonylureas (e.g. glyburide, glimepiride, glipyride, glipizide, chlorpropamide, gliclazide, glisoxepid, acetohexamide, glibornuride, tolbutamide, tolazamide, carbutamide, gliquidone, glyhexamide, phenbutamide, tolcyclamide, etc.), biguanides (e.g. metformin, phenformin, buformin, etc.), glucagon antagonists (e.g. a peptide or non-peptide glucagon antagonist), glucosidase inhibitors (e.g. acarbose, miglitol, etc.), insulin secetagogues, insulin sensitizers (e.g. troglitazone, rosiglitazone, pioglitazone, etc.) and the like; or anti-obesity agents (e.g. sibutramine, orlistat, etc.) and the like. The compounds and compositions of the present invention and the other anti-diabetic agents or anti-hyperglycemic agents may be administered simultaneously, sequentially or separately.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, cupric, cuprous, ferric, ferrous, lithium, magnesium, manganic, manganous, potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthetic amines. Other pharmaceutically acceptable organic non-toxic bases from which salts can be formed include, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salts can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric, methanesulfonic, and tartaric acids.

The pharmaceutical compositions of the present invention comprise a compound of Formula (I), or a pharmaceutically acceptable salt thereof, as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, as well as administration through inhaling, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

The pharmaceutical compositions according to the invention are preferably adapted for oral administration.

In practice, the compounds of Formula (I), or pharmaceutically acceptable salts thereof, can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound of Formula (I), or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound of Formula (I), or a pharmaceutically acceptable salt thereof. The compounds of Formula (I), or pharmaceutically acceptable salts thereof, can also be included in pharmaceutical compositions in combination with one or more other therapeutically active compounds.

The pharmaceutical compositions of this invention include a pharmaceutically acceptable liposomal formulation containing a compound of Formula (I), or a pharmaceutically acceptable salt thereof.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent or other such excipient. These excipients may be, for example, inert diluents such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example, magnesium stearate, stearic acid or talc. The tablets may be uncoated or they may be coated by known techniques to delay disintegration and absorption in the gastrointestinal tract and thereby provide a sustained action over a longer time. For example, a time delay material such as glyceryl monostearate or glyceryl distearate may be used.

In hard gelatin capsules, the active ingredient is mixed with an inert solid diluent, for example, calcium carbonate, calcium phosphate or kaolin. In soft gelatin capsules, the active ingredient is mixed with water or an oil medium, for example, peanut oil, liquid paraffin or olive oil. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient.

For example, a formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound of Formula (I), or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

Pharmaceutical compositions of this invention can be in a form suitable for inhaled administration. Such administration can be in forms and utilizing carriers described in, for example, Particulate Interactions in Dry Powder Formulations for Inhalation, Xian Zeng et al, 2000, Taylor and Francis; Pharmaceutical Inhalation Aerosol Technology, Anthony Hickey, 1992, Marcel Dekker; and Respiratory Drug Delivery, 1990, Editor: P. R. Byron, CRC Press.

In addition to the aforementioned carrier ingredients, the pharmaceutical compositions described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of Formula (I), or a pharmaceutically acceptable salt thereof, may also be prepared in powder or liquid concentrate form.

Generally, dosage levels of the order of from about 0.01 mg/kg to about 150 mg/kg of body weight per day are useful in the treatment of the above-indicated conditions, or alternatively about 0.5 mg to about 10 g per patient per day. For example, diabetes may be effectively treated by the administration of from about 0.01 to 100 mg of the compound per kilogram of body weight per day, or alternatively about 0.5 mg to about 7 g per patient per day.

It is understood, however, that the specific dose level for any particular patient will depend upon a variety of factors including the age, body weight, general health, sex, diet, time of administration, route of administration, rate of excretion, drug combination and the severity of the disease in the particular diabetic patient undergoing therapy. Further, it is understood that the compounds and salts thereof of this invention can be administered at subtherapeutic levels prophylactically in anticipation of a hyperglycemic condition.

The compounds of Formula (I) may exhibit advantageous properties compared to known glucokinase activators, e.g. as illustrated in the assays described herein. In particular compounds of the invention may exhibit improved values for Kₘ, Vₘₐₓ, EC₅₀, maximum activation (glucose concentration=5 mM), and/or maximum blood glucose reduction on basal blood glucose levels (e.g. in C57BL/6J mice), or other advantageous pharmacological properties, compared to known GK activators

In accordance with this invention, the compounds of Formula (Ia) can be prepared following the protocol illustrated in Scheme 1 below:

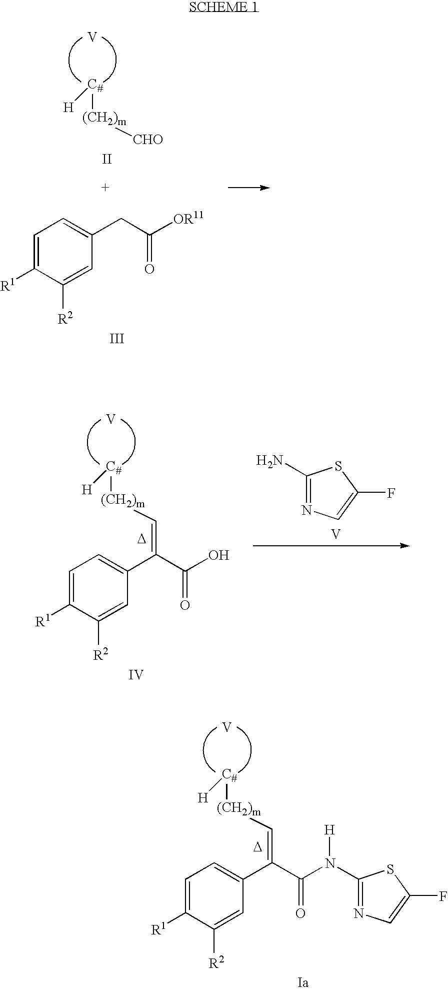

wherein V, R¹, R², m and Δ are as described above, and R¹¹ is C₁₋₄alkyl. The aldehydes II and phenylacetic esters III are commercially available or are readily prepared using known techniques. The α-carbanion of the phenylacetic ester III (R¹¹═C₁₋₄alkyl), generated at −78° C. in, for example, tetrahydrofuran, by a strong base, e.g. lithium diisopropylamide, may be condensed with II to give an α,β-unsaturated ester (T. Severin et al. Chem. Ber. 1985, 118, 4760-4773) that may be saponified using, for example, sodium hydroxide (W. L. Corbett et al., WO2001/44216), to produce IV. If necessary, any functional groups within the intermediate compounds, e.g. oxo or hydroxy groups in the compounds of formula II, may be protected and the protecting groups removed using conventional means. For example oxo groups may be protected as ketals and hydroxy groups as ethers, e.g. methoxymethyl (MOM) ethers.

The α,β-unsaturated carboxylic acids IV may be condensed with 2-amino-5-fluorothiazole V, or a salt thereof e.g. the hydrochloride salt, which may be prepared as described in the examples, using a variety of coupling conditions, e.g. polymer supported carbodiimide-1-hydroxybenzotriazole in N,N-dimethylformamide at 20° C. (for representative procedures, see http://www.argotech.com/PDF/resins/ps_carbodiimide.pdf and available from Argonaut Technologies, Inc., Foster City, Calif.), to give (Ia).

In accordance with this invention, the compounds of Formula (Ib) can be prepared following the protocol illustrated in Scheme 2 below:

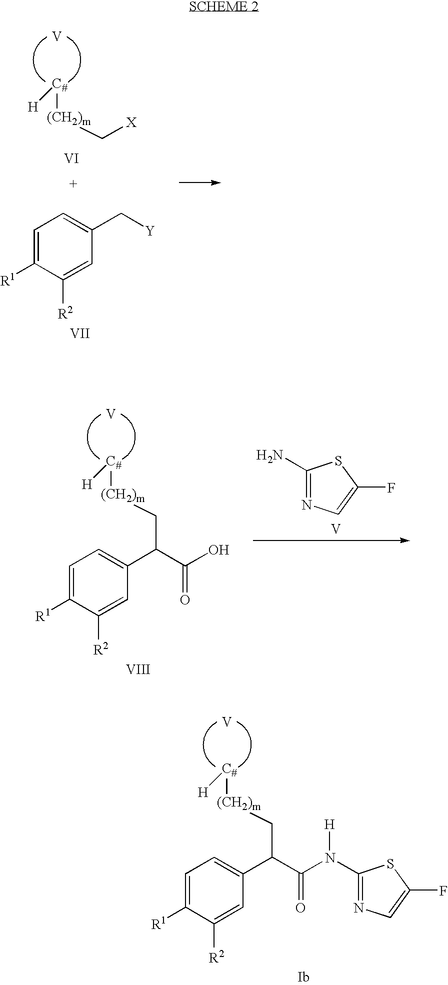

wherein V, R¹, R² and m are as described above, Y is CO₂R¹² wherein R¹² is hydrogen, C₁₋₄alkyl or benzyl; and X is chloro, bromo, iodo, or —OSO₂R¹³, wherein R¹³ is C₁₋₄alkyl, optionally substituted with one or more fluorines, or optionally substituted aryl.

The halides and sulfonate esters VI and the phenylacetic acids and esters VII are commercially available or are readily prepared using known techniques, for example as described in International Patent Publication Nos. WO2000/058293, WO2001/044216 and WO2003/095438. These alkylating agents may be reacted with the dianions of the phenylacetic acids VII, generated at −78° C. in tetrahydrofuran with ≧2 equivalents of a strong base, such as lithium diisopropylamide, to generate VIII directly (F. T. Bizzarro et al., WO2000/58293). Alternatively, the α-carbanion of phenylacetic ester VII, generated at −78° C. in tetrahydrofuran by a strong base, such as lithium bis(trimethylsilyl)amide (L. Snyder et al., J. Org. Chem. 1994, 59, 7033-7037), can be alkylated by VI to give α-substituted esters. Saponification of these esters, employing, for example, sodium hydroxide in aqueous methanol at 20° C. to reflux, leads to the carboxylic acids VIII. If necessary, any functional groups within the intermediate compounds, e.g. oxo or hydroxy groups in the compounds of formula VI, may be protected and the protecting groups removed using conventional means. For example oxo groups may be protected as ketals and hydroxy groups as ethers, e.g. methoxymethyl (MOM) ethers.

The carboxylic acids VIII may be condensed with 2-amino-5-fluorothiazole V, or a salt thereof e.g. the hydrochloride salt, which may be prepared as described in the examples, using a variety of coupling conditions, e.g. polymer supported carbodiimide-1-hydroxybenzotriazole in N,N-dimethylformamide at 20° C. (for representative procedures, see http://www.argotech.com/PDF/resins/ps_carbodiimide.pdf and available from Argonaut Technologies, Inc., Foster City, Calif.), to give amides (Ib).

The compound of Formula (Ib) has an asymmetric carbon atom which interlinks the amide carbonyl carbon, the aryl ring, and the —HC<>V containing sidechain. In accordance with this invention, the preferred stereoconfiguration at the asymmetric centre is (R).

If one desires to isolate the pure (R)— or (S)-stereoisomers of the compound of Formula (Ib), it is possible to resolve a racemic mixture of the chiral carboxylic acid precursor VIII by any conventional chemical means and then condense the enantiopure carboxylic acids with 2-amino-5-fluorothiazole V, or a salt thereof, using a reagent that causes negligible racemisation. By way of illustration, racemic VIII can be condensed with a chiral oxazolidinone derivative (see, for instance, F. T. Bizzarro et al. WO2000/58293) to generate a mixture of diastereoisomeric imides that are separable by any conventional method, e.g. column chromatography. Hydrolysis of the pure imides affords the stereopure (R)— and (S)-carboxylic acids that can then be condensed with 2-amino-5-fluorothiazole V, or a salt thereof, employing a reagent that minimises racemisation of the chiral centre, e.g. benzotriazol-1-yloxytris(pyrrolidino)phosphonium hexafluorophosphate (J. Coste et al. Tetrahedron Lett. 1990, 31, 205-208), to furnish enantiopure (R)— or (S)-amides of Formula (Ib). Alternatively, a racemic mixture of amides of Formula (Ib) can be separated by means of chiral high performance liquid chromatography employing a chiral stationary phase which can be purchased from, for example, Daicel Chemical Industries, Ltd, Tokyo, Japan.

Various functional groups present in the compounds of Formula (I) and intermediates for use in the preparation thereof may be produced by functional group conversions known to those skilled in the art. For example in the compounds of formula VIII sulfonyl groups may be produced by oxidation of the corresponding sulfanyl group using e.g. mCPBA.

Further details for the preparation of the compounds of Formula (I) are found in the examples.

The compounds of Formula (I) may be prepared singly or as compound libraries comprising at least 2, for example 5 to 1,000, compounds and more preferably 10 to 100 compounds of Formula (I). Compound libraries may be prepared by a combinatorial “split and mix” approach or by multiple parallel synthesis using either solution or solid phase chemistry, using procedures known to those skilled in the art.

During the synthesis of the compounds of Formula (I), labile functional groups in the intermediate compounds, e.g. hydroxy, oxo, carboxy and amino groups, may be protected. The protecting groups may be removed at any stage in the synthesis of the compounds of Formula (I) or may be present on the final compound of Formula (I). A comprehensive discussion of the ways in which various labile functional groups may be protected and methods for cleaving the resulting protected derivatives is given in, for example, Protective Groups in Organic Chemistry, T. W. Greene and P. G. M. Wuts, (1991) Wiley-Interscience, New York, 2^{nd} edition.

Any novel intermediates as defined above are also included within the scope of the invention. Thus the invention also provides:

a) a compound of formula W as defined above, wherein R¹ is SO₂R³, or SO₂NR⁴R⁵;

R² is hydrogen;

R³ is a C₁₋₃alkyl group, a C₃₋₇cycloalkyl group or a 4-6-membered heterocyclic group;

R⁴ and R⁵ are independently hydrogen or C¹⁻⁴alkyl, provided that R⁴ and R⁵ are not both hydrogen;

m is 0; and
Δ indicates that the double bond has the (E)-configuration; and

b) a compound of formula VIII as defined above, wherein R¹ is SO₂R³, or SO₂NR⁴R⁵;

R² is hydrogen;

R³ is a C₃₋₇cycloalkyl group or a 4-6-membered heterocyclic group;

R⁴ and R⁵ are independently hydrogen or C₁₋₄alkyl, provided that R⁴ and R⁵ are not both hydrogen; and

m is 0.

All publications, including, but not limited to, patents and patent application cited in this specification, are herein incorporated by reference as if each individual publication were specifically and individually indicated to be incorporated by reference herein as fully set forth.

### EXAMPLES

### Materials and Methods:

Column chromatography may be carried out on SiO₂ (40-63 mesh) unless specified otherwise. LCMS data may be obtained employing one of two methods: Method A: Waters Symmetry 3.5μ C₁₈ column (2.1×30.0 mm, flow rate=0.8 mL/min) eluting with a (5% MeCN in H₂O)-MeCN solution containing 0.1% HCO₂H over 6 min and V detection at 220 nm. Gradient information: 0.0-1.2 min: 100% (5% MeCN in H₂O); 1.2-3.8 min: Ramp up to 10% (5% MeCN in H₂O)-90% MeCN; 3.8-4.4 min: Hold at 10% (5% MeCN in H₂O)-90% MeCN; 4.4-5.5 min: Ramp up to 100% MeCN; 5.5-6.0 min: Return to 100% (5% MeCN in H₂O). Method B: Phenomenex Mercury Luna 3μ C₁₈ column (2.0×10.0 mm, flow rate=1.5 mL/min), eluting with a (5% MeCN in H₂O)-MeCN solution (4:1 to 1:4) containing 0.1% HCO₂H over 2.95 min, & employing diode array detection. The mass spectra for both Methods A and B may be obtained employing an electrospray ionisation source in either the positive (ES⁺) ion or negative ion (ES⁻) mode. Atmospheric Pressure Chemical Ionisation (APCI) spectra may be obtained on a FinniganMat SSQ 7000C instrument.

The synthesis of the following compound has been reported previously: 7(S)-iodomethyl-2(S),3(S)-diphenyl-1,4-dioxaspiro[4,4]nonane: WO2003/095438.

Abbreviations and acronyms: Ac: Acetyl; ATP: Adenosine 5′-triphosphate; n-Bu: n-Butyl; DMF: N,N-Dimethylformamide; DMPU: 1,3-Dimethyl-3,4,5,6-tetrahydro-2(1H)-pyrimidinone; DMSO: Dimethylsulfoxide; EDCI: 1-(3-Dimethylaminopropyl)-3-ethylcarbodiimide hydrochloride; Et: Ethyl; FA: Fold activation; GK: Glucokinase; Glc: Glucose; G6P: Glucose-6-phosphate; G6PDH: Glucose-6-phosphate dehydrogenase; GST-GK: Glutathione S-transferase-Glucokinase fusion protein; IH: Isohexane; LHMDS: Lithium bis(trimethylsilyl)amide; Me: Methyl; NADP(H): β-Nicotinamide adenine dinucleotide phosphate (reduced); NBS: N-Bromosuccinimide; Ph: Phenyl; rt: room temperature; RT: Retention time; TFAA: Trifluoroacetic anhydride; THF: Tetrahydrofuran.

### Intermediates

### Preparation 1: 5-Fluorothiazol-2-ylamine hydrochloride

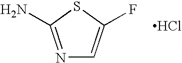

NEt₃ (63.4 mL, 455 mmol) was added to a stirred suspension of 5-bromothiazol-2-ylamine hydrobromide (102.7 g, 379 mmol) in CH₂Cl₂ (1.5 L). After 1 h, TFAA (64.2 mL, 455 mmol) was added dropwise at 0° C. over 15 min. The mixture was allowed to warm to 20° C. over 1 h, before being stirred for an additional 2 h. H₂O (600 mL) was added and the resulting precipitate was collected. The aqueous layer of the filtrate was separated and extracted with CHCl₃ (3×300 mL). The combined organic extracts were washed with brine, dried (Na₂SO₄), filtered and concentrated. The collected precipitate and residual solid were combined and triturated with EtOAc-n-C₆H₁₄ to give N-(5-bromothiazol-2-yl)-2,2,2-trifluoroacetamide: δ_{H} (CDCl₃): 7.45 (1H, s), 13.05 (1H, br). n-BuLi (253 mL of a 1.58M solution in hexanes, 403 mmol) was added dropwise over 50 min to a stirred solution of the above amide (50.0 g, 183 mmol) in anhydrous THF (1.3 L) at —78° C. After 1.5 h, a solution of N-fluorobenzenesulfonimide (86.0 g, 275 mmol) in anhydrous THF (250 mL) was added dropwise over 30 min. The mixture was stirred for 3 h, before being warmed up to −30° C. H₂O (300 mL) was added and the mixture was filtered through a Celite pad. The solid collected and Celite were washed with Et₂O (400 mL) and H₂O (400 mL). The organic layer of the filtrate was separated and extracted with water (2×400 mL). The combined aqueous layers were washed with Et₂O (400 mL), before being acidified to pH 6.5 with 2M HCl and extracted with EtOAc (2×400 mL). The combined organic extracts were washed with H₂O (2×400 mL) and brine, before being dried (MgSO₄), filtered and concentrated. Column chromatography (EtOAc-n-C₆H₁₄, 1:3 to 1:2) gave N-(5-fluorothiazol-2-yl)-2,2,2-trifluoroacetamide: δ_{H} (CDCl₃): 7.13 (1H, d). AcCl (12.6 mL, 175 mmol) was added dropwise to a stirred solution of this amide (15.7 g, 73 mmol) in MeOH (300 mL) at 0° C. The mixture was stirred at 20° C. for 30 min, heated under reflux for 1 h, and finally concentrated in vacuo. The residual solid was triturated with THF to give the title compound: δ_{II} (D₂O): 7.00 (1H, d).

The free base of the title compound was prepared by suspending the HCl salt in ether, washing with saturated aqueous NaHCO₃, drying the ethereal layer and evaporating to give the free base which was used immediately.

### Preparation 2: Ethyl (4-methanesulfonylphenyl)acetate

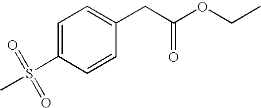

SOCl₂ (8.2 mL, 112.0 mmol) was added to a stirred suspension of (4-methanesulfonylphenyl)acetic acid (20.00 g, 93.3 mmol) in EtOH (80 mL) at −10° C. The mixture was allowed to warm up to 20° C. over 16 h, then the solvents were removed under reduced pressure. The remainder was dissolved in EtOAc and the resulting solution was washed with H₂O until the pH of the aqueous phase was neutral. The EtOAc solution was washed further with saturated aqueous Na₂CO₃, before being dried (MgSO₄). Filtration and solvent evaporation gave the title compound: m/z (ES⁺)=284.1 [M+MeCN+H]⁺.

Preparations 3-14: 2(R)-2-(3-chloro-4-methanesulfonylphenyl)-3-((R)-3-oxocyclopentyl)propionic acid, 2(R)-2-(3-chloro-4-methanesulfonylphenyl)-3-(4-oxocyclohexyl)propionic acid and 2(R)-2-(3-chloro-4-methanesulfonylphenyl)-3-(3-hydroxycyclopentyl)propionic acid may be prepared as described in WO2003/095438. The carboxylic acid intermediates of formula VIII required for the synthesis of Examples 7-15 may be prepared by the same general procedure, involving alkylation of the appropriate ester with 4-iodomethyl-HC<>V followed by hydrolysis of the product.

The carboxylic acid intermediate of formula VIII required for the synthesis of Example 7 was prepared as follows:

### Preparation 6a: (4-Cyclopropylsulfanylphenyl)oxoacetic acid

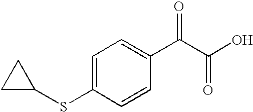

2M aqueous NaOH (163 mL) was added to a solution of ethyl (4-cyclopropylsulfanylphenyl)oxoacetate (40.62 g, 162.5 mmol) in EtOH (200 mL) and the stirred mixture heated at 60° C. for 2 h. After cooling, the mixture was concentrated to 150 mL and washed with ether (2×100 mL). Sufficient concentrated HCl was then added to adjust the pH to 1 and the resulting precipitate was extracted into EtOAc (2×300 mL). The combined organic phases were washed with water (3×100 mL), brine (200 mL) and dried (MgSO₄). Removal of the solvent gave the title compound: m/z (ES⁻)=221.0 [M−H⁺]⁻.

### Preparation 6b: (4-Cyclopropylsulfanylphenyl)acetic acid

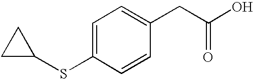

Hydrazine hydrate (14.19 g, 283.5 mmol) was cooled to −50° C. and (4-cyclopropylsulfanylphenyl)oxoacetic acid (Preparation 6a, 12.6 g, 56.7 mmol) added in one portion. The vigorously-stirred slurry was warmed firstly to rt and then at 80° C. for 5 min. Solid KOH (8.76 g, 156.5 mmol) was added in four equal portions and the resulting solution heated at 100° C. for 20 h. On cooling to rt, water (25 mL) was added and the aqueous phase washed with Et₂O (20 mL). The ethereal phase was itself washed with water (2×15 mL) and sufficient concentrated HCl added to the combined aqueous phases to adjust the pH to 1. The resulting precipitate was then extracted into EtOAc (2×300 mL) and the combined organic phases washed with water (3×100 mL), brine (200 mL) then dried (MgSO₄). Evaporation of the solvent gave the title compound: m/z (ES⁻)=207.1 [M−H⁺]⁻.

### Preparation 6c: 2-(4-Cyclopropylsulfanylphenyl)-N-(2(R)-hydroxy-1(R)-methyl-2-phenylethyl)-N-methylacetamide

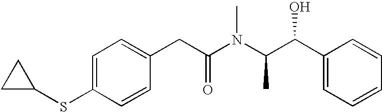

Anhydrous acetone (148 mL) was added to (4-cyclopropylsulfanylphenyl)-acetic acid (Preparation 6b, 16.41 g, 78.8 mmol) and K₂CO₃ (32.67 g, 236.4 mmol) to form a slurry which was cooled to −10° C. with stirring. Neat trimethylacetyl chloride (10.2 mL, 82.74 mmol) was introduced dropwise, ensuring the temperature did not exceed −10° C. during the addition. The reaction mixure was stirred at −10° C. for 20 min, warmed to 0° C. for 20 min then cooled to −15° C. and solid (1(R),2(R))-(−)-pseudoephedrine (19.53 g, 118.2 mmol) was added in one portion. After 10 min, the reaction mixture was brought to rt, where stirring was continued for 1.5 h. Water (100 mL) was added and the mixture extracted with EtOAc (500 mL). The organic phase was washed with water (2×100 mL) and the combined aqueous layers back-extracted with EtOAc (2×250 mL). The combined organic layers were then washed with brine (100 mL) and dried (MgSO₄). The solvent was removed and the solid yellow residue recrystallized from EtOAc-IH to give the title compound: m/z (ES⁺)=356.1 [M+H]⁺.

### Preparation 6d: 2(R)-(4-Cyclopropylsulfanylphenyl)-3-(3(R)-oxocyclopentyl)propionic acid

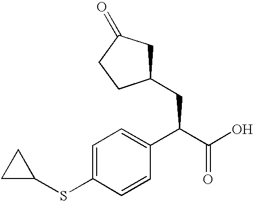

LHMDS (162 mL of a 1M solution in THF, 162 mmol) was diluted with anhydrous THF (161 mL) and cooled to −20° C. with stirring. A solution of 2-(4-cyclopropylsulfanylphenyl)-N-(2(R)-hydroxy-1(R)-methyl-2-phenylethyl)-N-methylacetamide (Preparation 6c, 30 g, 84.4 mmol) in anhydrous THF (245 mL) was added via cannula over 10 min, ensuring the reaction temperature remained below −15° C. throughout the addition. The reaction was allowed to warm to −7° C. over 30 min then cooled to −12° C. and a solution of 7(S)-iodomethyl-2(S),3(S)-diphenyl-1,4-dioxaspiro[4,4]nonane (27 g, 64.2 mmol) in a mixture of anhydrous THF (111 mL) and DMPU (18.9 mL) added via cannula over 10 min, ensuring the reaction temperature remained below −7° C. throughout. The reaction was warmed to 2° C. and stirred for 4.5 h before being poured into a mixture of toluene (770 mL) and 20% aqueous NH₄Cl (550 mL). After stirring vigorously, the organic layer was separated and washed with 20% aqueous NH₄Cl (550 mL) and brine (100 mL). The aqueous phases were combined and extracted with EtOAc (500 mL) which, after separation, was washed with brine (100 mL). The combined organic phases were dried (MgSO₄), filtered, evaporated and the resulting oil purified by flash chromatography (IH-EtOAc, 9:1 changing incrementally to 1:1) to give 2(R)-(4-cyclopropylsulfanylphenyl)-3-(2(S),3(S)-diphenyl-1,4-dioxaspiro[4.4]non-7(R)-yl)-N-(2(R)-hydroxy-1(R)-methyl-2-phenylethyl)-N-methylpropionamide: m/z (ES⁺)=648.3 [M+H]⁺. A stirred solution of this amide (30.7 g, 47.38 mmol) in 1,4-dioxane (62 mL) was diluted with 4.5M aqueous H₂SO₄ (61.5 mL) and the resulting mixture heated under gentle reflux for 18 h. After cooling on ice, water (162 mL) was added and the mixture extracted with EtOAc (250 mL). The aqueous layer was separated and extracted further with EtOAc (2×150 mL) and the combined organic phases washed with water (3×200 mL), ensuring the final wash was pH neutral, and brine (100 mL). After drying (MgSO₄) and filtering, the solvent was removed and the residue purified by flash chromatography (CH₂Cl₂ then CH₂Cl₂-THF, 5:1 changing to 3:1) to give the title compound: m/z (ES⁺)=305.1 [M+H]⁺.

### Preparation 6e: 2(R)-(4-Cyclopropanesulfonylphenyl)-3-(3(R)-oxocyclopentyl)propionic acid

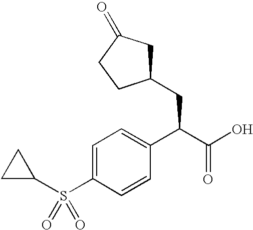

A stirred solution of 2(R)-(4-cyclopropylsulfanylphenyl)-3-(3(S)-oxocyclopentyl)propionic acid (Preparation 6d, 5.0 g, 16.43 mmol) in CH₂Cl₂ (250 mL) was cooled to 1° C. on ice and 70% mCPBA (8.099 g, 32.85 mmol) added portionwise, maintaining the temperature below 3° C. After 6 h the solvent was removed and the residue purified by flash chromatography (1% AcOH in CH₂Cl₂ then THF) to give the title compound: m/z (ES⁺)=337.1 [M+H]⁺.

### Preparations 15-17

The intermediates of formula IV required for the synthesis of Examples 4-6 may be prepared by the following general processes. Where necessary, any functional groups within the intermediate compounds, e.g. oxo or hydroxy groups in the compounds of formula II, may be protected and the protecting groups removed using conventional means:

Method A: LDA (24 mL of a 1.8M solution in n-C₇H₁₆-THF-PhEt, 43.3 mmol) is added dropwise to a stirred solution of DMPU (19 mL, 153.0 mmol) in anhydrous THF (100 mL) at −78° C. After 30 min, a solution of the appropriate phenylacetic ester III (20.6 mmol) in anhydrous THF (42 mL) is added dropwise. The mixture is stirred further for 1 h, before treating dropwise with a solution of aldehyde II or a protected derivative thereof (20.6 mmol) in anhydrous THF (25 mL). After being allowed to warm up to 20° C. over 16 h, the reaction is quenched with saturated aqueous NH₄Cl (210 mL). The THF is removed under reduced pressure, then the remainder is extracted with EtOAc (3×250 mL). The combined EtOAc extracts are dried (MgSO₄), filtered, and concentrated. Column chromatography furnishes the acrylate ethyl ester. This ester is saponified, for example, by heating a solution of this ester (19.1 mmol) in MeOH (30 mL) and 1M NaOH (40 mL, 40.0 mmol) under reflux for 1 h. On cooling, the mixture is washed with EtOAc. The aqueous phase is acidified with 1M HCl, before being extracted with EtOAc. The combined organic extracts are dried (MgSO₄). Filtration and solvent evaporation affords the desired (E)-acrylic acid.

Method B: NaOEt (0.63 mL of a 0.5M solution in EtOH, 0.32 mmol) is added dropwise to a stirred solution of phenylacetic ester III (3.16 mmol) and aldehyde II or a protected derivative thereof (3.47 mmol) in anhydrous DMSO (3 mL). The mixture is heated at 80° C. for 16 h, before being treated with AcOH to adjust the pH to 7. EtOAc (30 mL) is added, then the solution is washed with H₂O (2×10 mL) and brine (10 mL), before being dried (MgSO₄). Filtration, solvent evaporation, and column chromatography yields the acrylate ethyl ester. This ester is saponified as described above in Method A to give the desired (E)-acrylic acid.

### EXAMPLES

The following compounds may be made using the general methods described below:

| Example | Structure | Name |
|---|---|---|
| 1 | 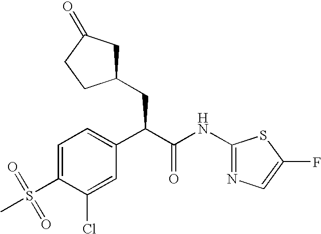 | 2(R)-2-(3-Chloro-4-methanesulfonylphenyl)-N-(5-fluorothiazol-2-yl)-3-((R)-3-oxocyclopentyl)propionamide |
| 2 | 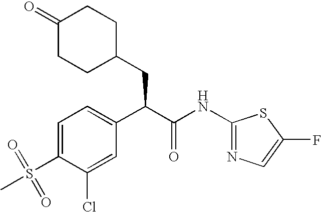 | 2(R)-2-(3-Chloro-4-methanesulfonylphenyl)-N-(5-fluorothiazol-2-yl)-3-(4-oxocyclohexyl)propionamide |
| 3 | 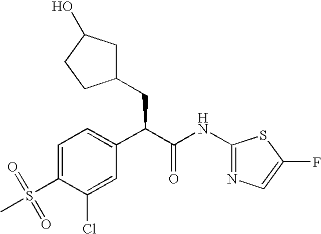 | 2(R)-2-(3-Chloro-4-methanesulfonylphenyl)-N-(5-fluorothiazol-2-yl)-3-(3-hydroxycyclopentyl)propionamide |
| 4 | 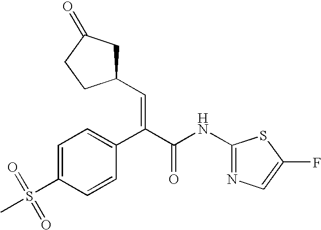 | (E)-N-(5-Fluorothiazol-2-yl)-2-(4-methanesulfonylphenyl)-3-((S)-3-oxocyclopentyl)acrylamide |
| 5 | 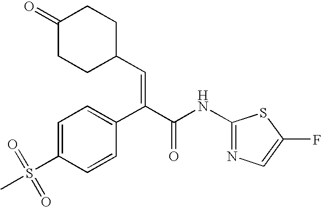 | (E)-N-(5-Fluorothiazol-2-yl)-2-(4-methanesulfonylphenyl)-3-(4-oxocyclohexyl)acrylamide |
| 6 | 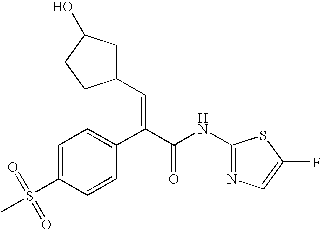 | (E)-N-(5-Fluorothiazol-2-yl)-3-(3-hydroxycyclopentyl)-2-(4-methanesulfonylphenyl)acrylamide |
| 7 | 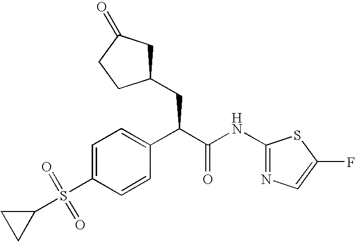 | 2(R)-2-(4-Cyclopropanesulfonylphenyl)-N-(5-fluorothiazol-2-yl)-3-((R)-3-oxocyclopentyl)propionamide |
| 8 | 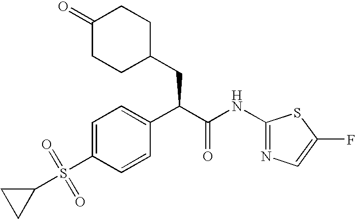 | 2(R)-2-(4-Cyclopropanesulfonylphenyl)-N-(5-fluorothiazol-2-yl)-3-(4-oxocyclohexyl)propionamide |
| 9 | 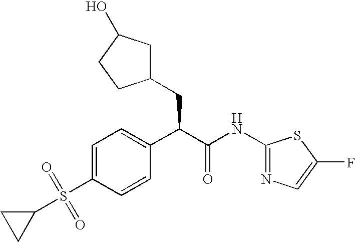 | 2(R)-2-(4-Cyclopropanesulfonylphenyl)-N-(5-fluorothiazol-2-yl)-3-(3-hydroxycyclopentyl)propionamide |
| 10 | 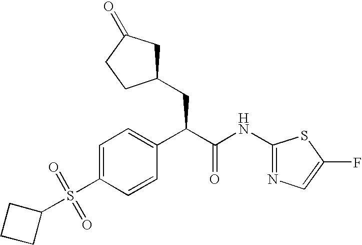 | 2(R)-2-(4-Cyclobutanesulfonylphenyl)-N-(5-fluorothiazol-2-yl)-3-(3-oxocyclopentyl)propionamide |
| 11 | 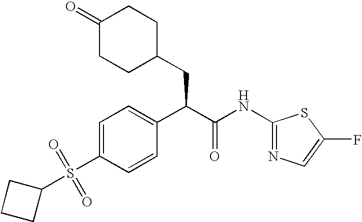 | 2(R)-2-(4-Cyclobutanesulfonylphenyl)-N-(5-fluorothiazol-2-yl)-3-(4-oxocyclohexyl)propionamide |
| 12 | 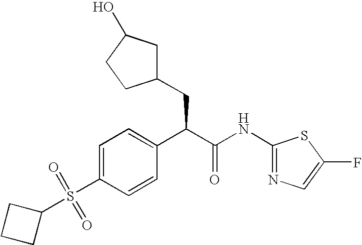 | 2(R)-2-(4-Cyclobutanesulfonylphenyl)-N-(5-fluorothiazol-2-yl)-3-(3-hydroxycyclopentyl)propionamide |
| 13 | 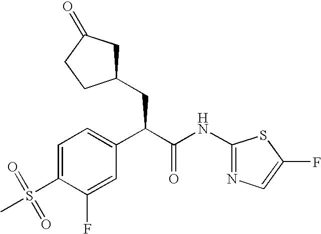 | 2(R)-2-(3-Fluoro-4-methanesulfonylphenyl)-N-(5-fluorothiazol-2-yl)-3-((R)-3-oxocylopentyl)propionamide |
| 14 | 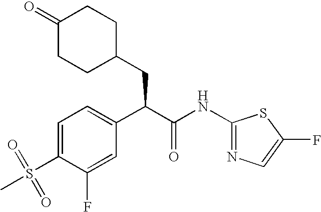 | 2(R)-2-(3-Fluoro-4-methanesulfonylphenyl)-N-(5-fluorothiazol-2-yl)-3-(4-oxocyclohexyl)propionamide |
| 15 | 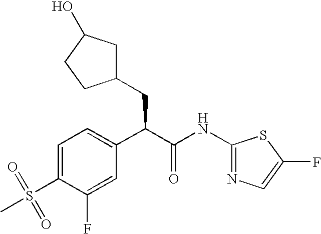 | 2(R)-2-(3-Fluoro-4-methanesulfonylphenyl)-N-(5-fluorothiazol-2-yl)-3-(3-hydroxycyclopentyl)propionamide |

Method C: To a stirred solution of PPh₃ (3.53 g, 13.4 mmol) in CH₂Cl₂ (70 mL) is added NBS (882 mg, 10.6 mmol) at 0° C. After 10 min, the appropriate compound of Formula IV or VIII (9.0 mmol) is added, then the mixture is stirred at 0° C. for 20 min, and then at 20° C. for 30 min. 5-Fluorothiazol-2-ylamine hydrochloride (933 mg, 9.3 mmol) and pyridine (2.2 mL, 18.8 mmol) are added at 0° C., then the mixture is stirred at 20° C. for 20 h. After solvent evaporation, the residue is partitioned between 5% aqueous citric acid (100 mL) and EtOAc (500 mL). The aqueous layer is further extracted with EtOAc (200 mL), then the combined organic layers are washed with H₂O and brine, before being dried (Na₂SO₄), filtered, and concentrated in vacuo. Chromatographic purification (CHCl₃-MeOH, 99:1) of the residue on Chromatorex® NH-DM1020 (Fuji Silysia Chemical, Ltd., Aichi-ken, Japan; see also http://www.fuiji-silysia.cojp/e-fl100dx.htm) gives the desired compound.

Method D: EDCI (80 mg, 420 μmol) and HOBt (56 mg, 420 μmol) are added to a stirred solution of the appropriate compound of Formula IV or VIII (320 μmol) in anhydrous DMF (6 mL). After 15 min, the solution is treated with 5-fluorothiazol-2-ylamine hydrochloride (38 mg, 380 μmol) and pyridine (61 μL, 760 μmol). The mixture is stirred at 20° C. for 16 h, before being concentrated under reduced pressure. The residue is partitioned between CH₂Cl₂ and saturated aqueous Na₂CO₃. The organic layer is washed with 1M HCl and dried (MgSO₄). Filtration and solvent evaporation gives the desired compound, which, if racemic, can be separated by chiral stationary phase HPLC. Method: CHIRAL CEL OJ® (Daicel Chemical Industries, Ltd., Tokyo, Japan), 10 cm ø×25 cm, MeOH (100%), 189 mL/min, UV 285 nm, 25° C.

Method E: Oxalyl chloride (0.23 mL, 0.47 mmol) is added to a stirred solution of the appropriate compound of Formula IV or VIII (0.42 mmol) in anhydrous CH₂Cl₂ (6 mL) at 0° C. Anhydrous DMF (50 μL) is added, then the mixture is stirred at 0° C. for 2 h. 5-Fluorothiazol-2-ylamine (151 mg, 1.28 mmol; obtained by partitioning the hydrochloride salt between Et₂O and saturated aqueous Na₂CO₃, separation of Et₂O layer, drying (MgSO₄), and solvent evaporation) and pyridine (69 μL, 0.85 mmol) are added, then the mixture is stirred at 0-5° C. for 16 h, before finally being allowed to warm to 20° C. and diluted with EtOAc (45 mL). The solution is washed with 1M HCl (2×20 mL) and saturated aqueous Na₂CO₃ (2×20 mL), before being dried (MgSO₄), filtered, and concentrated. Purification via chromatography furnishes the desired compound.

The compound of Example 7, 2(R)-2-(4-cyclopropanesulfonylphenyl)-N-(5-fluorothiazol-2-yl)-3-((R)-3-oxocyclopentyl)propionamide, was prepared as follows:

A solution of 2(R)-(4-cyclopropanesulfonylphenyl)-3-(3(R)-oxocyclopentyl)propionic acid (Preparation 6e, 893 mg, 2.65 mmol) in anhydrous CH₂Cl₂ (38 mL) was cooled to 0° C. and a solution of oxalyl chloride (0.408 g, 3.21 mmol) in anhydrous CH₂Cl₂ (2 mL) added dropwise, maintaining the temperature at 0° C. during the addition. Dry DMF (0.08 mL) was added and the reaction mixture stirred 2.5 h. A solution of 2-amino-5-fluorothiazole (Preparation 1, 345 mg, 2.92 mmol) in anhydrous CH₂Cl₂ (6 mL) was introduced slowly, followed by pyridine (0.53 mL, 5.31 mmol) and the mixture stirred at 0° C. for 2 h then at rt overnight. The solution was diluted with CH₂Cl₂ (150 mL) and washed with aqueous 5% w/v citric acid (2×30 mL), saturated aqueous NaHCO₃ (2×30 mL), water (50 mL) and brine (50 mL). The organic phase was dried (MgSO₄), evaporated and the residue purified by flash chromatography (IH-EtOAc, 3:2) to afford the title compound: RT=3.47 min; m/z (ES⁺)=437.1 [M+H]⁺.

### Assays

### In Vitro GK Activity:

Using a protocol similar to that described in WO2000/58293, GK activity may be assayed by coupling the production of G6P by GST-GK to the generation of NADPH with G6PDH as the coupling enzyme.

The GK assay is performed at 30° C. in a flat bottom 96-well assay plate from Costar with a final incubation volume of 100 μL. The assay buffer contains: 25 mM Hepes buffer (pH 7.4), 12.5 mM KCl, 5 mM D-Glc, 5 mM ATP, 6.25 mM NADP, 25 mM MgCl₂, 1 mM dithiothreitol, test compound or 5% DMSO, 3.0 unit/mL G6PDH, and 0.4 μL/mL GST-GK, derived from human liver GK. ATP, G6PDH, and NADP may be purchased from Roche Diagnostics. The other reagents are >98% pure and may be purchased from Kanto Chemicals. The test compounds are dissolved in DMSO, before being added to the assay buffer without ATP. This mix is preincubated in the temperature controlled chamber of a SPECTRAmax 250 microplate spectrophotometer (Molecular Devices Corporation, Sunnyvale, Calif.) for 10 min, then the reaction started by the addition of 10 μL ATP solution.

After starting the reaction, the increase in optical density (OD) at 340 nm is monitored over a 10 min incubation period as a measure of GK activity. Sufficient GST-GK is added to produce an increase in OD₃₄₀ over the 10 min incubation period in wells containing 5% DMSO, but no test compound. Preliminary experiments have established that the GK reaction is linear over this period of time, even in the presence of activators that produced an 8-fold increase in GK activity. The GK activity in control wells is compared with the activity in wells containing test GK activators. The compound concentrations that produced a 50% increase in GK activity (i.e. FA1.5) are calculated. GK activators achieve FA1.5 at ≦30 μM. Using a range of dilutions of the test compound, the maximum increase in GK activity can be calculated along with the concentration of test compound which produces 50% activation (EC₅₀).

The compound of Example 7 achieved greater than 4 fold maximum activation of GK and had an EC₅₀<0.5 μM.

In vivo GK Activity:

Following an 18 h fasting period, C57BL/6J mice are dosed orally via gavage with GK activator at 50 mg/kg body weight. Blood Glc determinations are made 5 times during the 6 h post-dose study period.

Mice (n=5) are weighed and fasted for 18 h before oral treatment. GK activators are dissolved in the Gelucire vehicle reported in WO 00/58293 (EtOH:Gelucire44/14:PEG400 q.s. 4:66:30 v/v/v) at a concentration of 13.3 mg/mL. Mice are dosed orally with 7.5 mL formulation per kg of body weight to equal a 50 mg/kg dose. Immediately prior to dosing, a pre-dose (time zero) blood Glc reading is acquired by snipping off a small portion of the animals' tails (<1 mm) and collecting 15 μL blood for analysis. After GK activator treatment, further blood Glc readings are taken at 1, 2, 4, and 6 h post-dose from the same tail wound. Results are interpreted by comparing the mean blood Glc values of 5 vehicle treated mice with the 5 GK activator treated mice over the 6 h study duration. Compounds are considered active when they exhibit a statistically significant decrease in blood Glc compared to vehicle for 2 consecutive assay time points.

1. A compound of Formula (I): 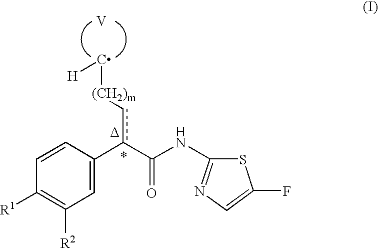
or a pharmaceutically acceptable salt thereof, wherein.
V is (CH₂)ₖ where one CH₂ group may optionally be replaced by CH(OH), C═O, C═NOH, C═NOCH₃, CHX, CXX¹, CH(OCH₃), CH(OCOCH₃), or C(OH)(C₁₋₄alkyl);
X and X¹ are independently selected from fluoro and chloro;
R¹ and R² are independently selected from hydrogen, halogen, hydroxy, amino, cyano, nitro, SR³, SOR³, SO₂R³, SO₂NR⁴R⁵, NHSO₂R³, or a C₁₋₄alkyl, C₂₋₄alkenyl, C₂₋₄alkynyl, C₁₋₄alkoxy, or heteroaryl group, wherein any group is optionally substituted with 1 to 5 substituents independently selected from halogen, cyano, nitro, hydroxy, C₁₋₂alkoxy, —N(C₀₋₂alkyl)(C₀₋₂alkyl), C₁₋₂alkyl, CFₙH₃₋ₙ, aryl, heteroaryl, —CON(C₀₋₂alkyl)(C₀₋₂alkyl), SCH, SOCH₃, SO₂CH_{3,} and —SO₂N(C₀₋₂alkyl)(C₀₋₂alkyl);
R³ is a C₁₋₄alkyl group, aryl group, heteroaryl group, or 4- to 7-membered heterocyclic group, wherein any group is optionally substituted with 1 to 5 substituents independently selected from halogen, cyano, nitro, hydroxy, C₁₋₂alkoxy, —N(C₀₋₂alkyl)(C₀₋₂alkyl), C₁₋₂alkyl, C₃₋₇cycloalkyl, 4- to 7-membered heterocyclic ring, CFₙH₃₋ₙ, aryl, heteroary, COC₁₋₂alkyl, —CON(C₀₋₂alkyl)(C₀₋₂alkyl), SOCH₃, SO₂CH₃, and —SO₂N(C₀₋₂alkyl)(C₀₋₂alkyl);
R⁴ and R⁵ are independently hydrogen, or a C₁₋₄alkyl group, C₃₋₇cycloalkyl group, aryl group, heteroaryl group, or 4- to 7-membered heterocyclic group, wherein any group is optionally substituted with 1 to 5 substituents independently selected from halogen, cyano, nitro, hydroxy, C₁₋₂alkoxy, —N(C₀₋₂alkyl)(C₀₋₂alkyl), C₁₋₂alkyl, C₃₋₇cycloalkyl, 4- to 7-membered heterocyclic ring, CFₙH₃₋ₙ, aryl, heteroaryl, —CON(C₀₋₂alkyl)(C₀₋₂alkyl), SOCH₃, SO₂CH₃, and —SO₂N(C₀₋₂alkyl)(C₀₋₂alkyl),
or R⁴ and R⁵ together form a 4- to 8-membered heterocyclic ring which is optionally substituted with 1 or 2 substituents independently selected from C₁₋₂alkyl and hydroxy;
k is an integer from 2 to 7;
m is 0 or 1;
n is 1, 2 or 3; and
the dotted line together with the solid line forms an optional double bond, and Δ indicates that the double bond has the (E)-configuration.

2. A compound according to claim 1, or a pharmaceutically acceptable salt thereof, wherein the dotted line together with the solid line forms a double bond.

3. A compound according to claim 1, or a pharmaceutically acceptable salt thereof, wherein the dotted line together with the solid line forms a single bond.

4. A compound according to claim 3, or a pharmaceutically acceptable salt thereof, wherein the dotted line together with the solid line forms a single bond, and the absolute configuration at the asymmetric centre α to the amide carbonyl carbon is (R).

5. A compound according to claim 1, or a pharmaceutically acceptable salt thereof, wherein m is 0.

6. A compound according to claim 1, or a pharmaceutically acceptable salt thereof, wherein k is 4 or 5.

7. A compound according to claim 6, or a pharmaceutically acceptable salt thereof, wherein the group formed by —HC< and >V represents 3-oxocyclopentyl, 4-oxocyclohexyl or 3-hydroxycyclopentyl.

8. A compound according to claim 1, or a pharmaceutically acceptable salt thereof, wherein R¹ and R² are not both hydrogen.

9. A compound according to claim 1, or a pharmaceutically acceptable salt thereof, wherein R¹ is SOR³, SO₂R³, or SO₂NR⁴R⁵.

10. A compound according to claim 1, or a pharmaceutically acceptable salt thereof, wherein R³ is C₁₋₄alkyl or C₃₋₇cycloalkyl.

11. A compound according to claim 1, or a pharmaceutically acceptable salt thereof, wherein R¹ is SO₂C₃₋₄cycloalkyl.

12. A compound according to claim 1, or a pharmaceutically acceptable salt thereof, wherein R² is hydrogen, chloro, fluoro, or trifluoromethyl.

13. A compound according to claim 1 selected from:
2(R)-2-(3-Chloro-4-methanesulfonylphenyl)-N-(5-fluorothiazol-2-yl)-3-((R)-3-oxocyclopentyl)propionamide;
2(R)-2-(3-Chloro-4-methanesulfonylphenyl)-N-(5-fluorothiazol-2-yl)-3-(4-oxocyclohexyl)propionamide;
2(R)-2-(3-Chloro-4-methanesulfonylphenyl)-N-(5-fluorothiazol-2-yl)-3-(3-hydroxycyclopentyl)propionamide;
(E)-N-(5-Fluorothiazol-2-yl)-2-(4-methanesulfonylphenyl)-3-((S)-3-oxocyclopentyl)acrylamide;
(E)-N-(5-Fluorothiazol-2-yl)-2-(4-methanesulfonylphenyl)-3-(4-oxocyclohexyl)acrylamide;
(E)-N-(5-Fluorothiazol-2-yl)-3-(3-hydroxycyclopentyl)-2-(4-methanesulfonylphenyl)acrylamide;
2(R)-2-(4-Cyclopropanesulfonylphenyl)-N-(5-fluorothiazol-2-yl)-3-((R)-3-oxocyclopentyl)propionamide;
2(R)-2-(4-Cyclopropanesulfonylphenyl)-N-(5-fluorothiazol-2-yl)-3-(4-oxocyclohexyl)propionamide;
2(R)-2-(4-Cyclopropanesulfonylphenyl)-N-(5-fluorothiazol-2-yl)-3-(3-hydroxycyclopentyl)propionamide;
2(R)-2-(4-Cyclobutanesulfonylphenyl)-N-(5-fluorothiazol-2-yl)-3-(3-oxocyclopentyl)propionamide;
2(R)-2-(4-Cyclobutanesulfonylphenyl)-N-(5-fluorothiazol-2-yl)-3-(4-oxocyclohexyl)propionamide;
2(R)-2-(4-Cyclobutanesulfonylphenyl)-N-(5-fluorothiazol-2-yl)-3-(3-hydroxycyclopentyl)propionamide;
2(R)-2-(3-Fluoro-4-methanesulfonylphenyl)-N-(5-fluorothiazol-2-yl)-3-((R)-3-oxocyclopentyl)propionamide;
2(R)-2-(3-Fluoro-4-methanesulfonylphenyl)-N-(5-fluorothiazol-2-yl)-3-(4-oxocyclohexyl)propionamide; and
2(R)-2-(3-Fluoro-4-methanesulfonylphenyl)-N-(5-fluorothiazol-2-yl)-3-(3-hydroxycyclopentyl)propionamide;
or a pharmaceutically acceptable salt of any one thereof.

14. A pharmaceutical composition comprising a compound according to claim 1, or a pharmaceutically acceptable salt thereof, and a pharmaceutically acceptable carrier.

15. A method of therapeutic treatment of a condition where activation of GK is desirable comprising a step of administering an effective amount of a compound according to claim 1, or a pharmaceutically acceptable salt thereof.

16. A method according to claim 15 wherein the condition where activation of GK is desirable is hyperglycemia or diabetes.

17. A method according to claim 16 wherein the compound according to claim 1 is administered in combination with one or more other anti-hyperglycemic agents or anti-diabetic agents.

18. A method according to claim 15 wherein the condition where activation of GK is desirable is pre-diabetic hyperglycemia or impaired glucose tolerance.

19. A process for the preparation of a compound of Formula (I): 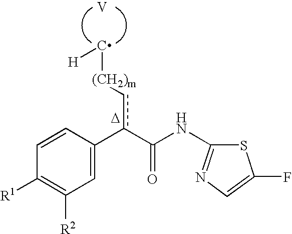
(I) or a pharmaceutically acceptable salt thereof, said process comprising the condensation of a compound immediately below: 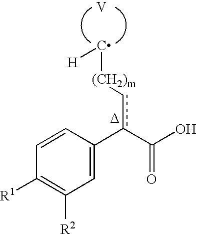
with a compound of Formula (V): 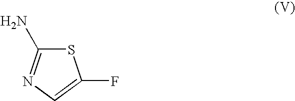
or a salt thereof, wherein the dotted line together with the solid line forms an optional double bond and V, R¹, R², m and Δ are as defined in claim 1.

20. (canceled)

21. A compound of a formula illustrated below: 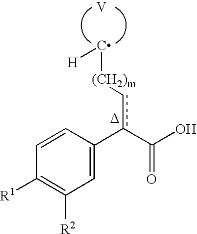
wherein the groups formed by —HC< and >V represents oxocycloalkyl or hydroxycycloalkyl;
R¹ is SO₂R³, or SO₂NR⁴R⁵;
R² is hydrogen;
R³ is a C₃₋₇cycloalkyl group or a 4- to 6-membered heterocyclic group;
R⁴ and R⁵ are independently hydrogen or C₁₋₄alkyl, provided that R⁴ and R⁵ are not both hydrogen;
m is 0; and
the dotted line together with the solid line forms an optional double bond, and Δ indicates that the double bond has the (E)-configuration.

22. (canceled)

23. A compound according to claim 21 wherein the dotted line together with the solid line forms a double bond.

24. A compound according to claim 21 wherein the dotted line together with the solid line forms a single bond.

25. The process according to claim 19 wherein the dotted line together with the solid line forms a single bond.

26. The process according to claim 19 wherein the dotted line together with the solid line forms a double bond.
